(19) United States
(12) Patent Application Publication
(10) Pub. No.: US 2022/0051802 A1
(43) Pub. Date: Feb. 17, 2022

(54) SYSTEM AND METHOD FOR DETECTION AND MONITORING OF OVER SEDATION TO PREVENT HARM TO PATIENTS

(71) Applicant: DIGNITY HEALTH, San Francisco, CA (US)

(72) Inventors: Candace FONG, Phoenix, AZ (US); Ingrid WURPTS, Phoenix, AZ (US); Ken FERRELL, Phoenix, AZ (US); Crestina BRACKEEN, Phoenix, AZ (US); Julie VAILS, Phoenix, AZ (US); Alyson D'ANDREA, Phoenix, AZ (US); Angelica Marie CHANCO-LARIOS, Phoenix, AZ (US); Gurmeet SRAN, Phoenix, AZ (US); Joseph COLORAFI, Phoenix, AZ (US); Tim MELDEN, Phoenix, AZ (US); Michael SIECZKA, Phoenix, AZ (US); Sunilkumar KAKADE, Phoenix, AZ (US); Mark PAGE, Phoenix, AZ (US); Arlene PYNADATH, Phoenix, AZ (US); Gaurav PADIYAR, Phoenix, AZ (US); Umesh KALYANASUNDARAM, Phoenix, AZ (US); Murali NANDULA, Phoenix, AZ (US); Monica SPOERER, Phoenix, AZ (US); Harry SCHNED, Phoenix, AZ (US)

(21) Appl. No.: 17/312,827

(22) PCT Filed: Dec. 11, 2019
(86) PCT No.: PCT/US2019/065763

§ 371 Date: Jun. 10, 2021

Related U.S. Application Data
(60) Provisional application No. 62/778,979, filed on Dec. 13, 2018.

(51) Int. Cl. G16H 50/30; G16H 10/60; G16H 20/10

(52) U.S. Cl. CPC G16H 50/30; G16H 20/10; G16H 10/60

(57) ABSTRACT

A system and method for identifying over sedation risk factors and generating appropriate alerts for healthcare professionals as a result of an algorithm triggered based whether a calculated over sedation risk score/level for the patient crosses a predefined threshold, and determine whether the patient score is at a low, moderate, or high risk for over sedation. If the patient is at a moderate or high risk, an alert may be triggered and displayed to the healthcare professional, along with data associated with the alert in a synchronized timeline displayed on a user interface.

### BACKGROUND

Sedation of patients provides relief from pain. However, patients may become over sedated, which may require the reversal of the sedation, using reversal agents such as naloxone or flumazenil. This may also cause complications in the healing process and may create additional costs for healthcare professionals. The over sedation of patients may also represent a more significant problem: depending on the medical history of the patient, over sedation may cause irreparable harm to them physically. To compound these complications, America is currently undergoing an opioid crisis, which has created additional focus on ensuring that the administration of sedation medication strikes the balance between alleviating the pain of the patients, while making sure that the amount of sedation is not more than the patient needs.

Even before the opioid crisis, various healthcare organizations have observed over sedation with regard to their patients. The patients that were over sedated showed symptoms of respiratory depression from the over sedation, which resulted in the patients being unable to breathe, which, in turn, resulted in anoxic brain injury or even death. Other negative side effects of over sedation include encephalopathy, respiratory arrest, cardiac arrest, aspiration pneumonia, and neurological injury.

However, the current state of the art does not include a system that can alert the doctors, nurses, or other healthcare professionals, referred to as providers herein to be proactive in determining whether or not to provide patients with additional doses of sedation medication, while also considering the effects of over sedation.

### SUMMARY

The present disclosure overcomes the aforementioned drawbacks by providing a system that can alert healthcare professionals of such dangers, and encourage them to be proactive in providing an appropriate amount of sedative medication, while avoiding administration of additional sedatives that would cause over sedation. This may reduce or eliminate unintended over sedation and the need for reversal agents in order to reverse the effects of opioids and/or benzodiazepines.

Using literature parameters, generated models, and/or identified risk factors, the disclosed system may generate an alert for healthcare professionals (e.g., nurses or physicians). The alert may be generated as a result of an algorithm that incorporates various risk factors identified within existing medical literature and associated with a patient (e.g., age, sleep apnea issues, respiratory issues, whether or not they are a smoker, renal and liver factors, etc.), and uses these risk factors to generate a risk score, used to trigger the algorithm, warning the healthcare professionals of the patient's risk of over sedation.

The disclosed system may execute the algorithm, triggered based on a determination of whether a patient reaches a certain threshold, and whether the calculated risk score/level for the patient crosses that threshold. The disclosed system may further include several additional thresholds, used to determine whether the patient score is at a low, moderate, or high risk for over sedation. If the patient is at a moderate or high risk, an alert may be triggered and displayed to the healthcare professional, alerting them that the patient is at a heightened risk.

For example, if a patient is complaining about pain, and they're about to receive an extra dose of the medication, the disclosed system may generate a risk score, determine if the patient is at a moderate or high risk of over sedation based on the risk score, and generate an alert, which may be displayed to the healthcare professional, allowing them to consider all factors before administering the next dose of the sedative, thereby avoiding the possibility of over sedation. Because the provider is alerted to the risk of over sedation, the disclosed dashboard may act as a type of “yellow light,” reminding the provider to stop, review the history of the patient and any healthcare issues or additional medication given using a user interface disclosed herein, and assess the patient before administering the medication, thereby avoiding over sedation if the extra dose of sedation medication is given.

### BRIEF DESCRIPTION OF THE DRAWINGS

FIG. 1 is a block diagram of a system configured to implement the present disclosure.

FIG. 2A is a flow chart setting forth the steps of processes for extracting data from a data repository, utilizing the extracted data in generating risk scores and levels for users, generating an alert, and displaying relevant data on a user interface in near real time, in accordance with the present disclosure.

FIG. 2B is a more detailed flow chart setting forth the steps of calculating a machine learning model based risk score, in accordance with the present disclosure.

FIG. 2C is a more detailed flow chart setting forth the steps of calculating an alternative risk score, in accordance with the present disclosure.

FIG. 2D is a more detailed flow chart setting forth additional steps of calculating an alternative risk score, in accordance with the present disclosure.

FIG. 3 is a non-limiting example interface used in displaying data and monitoring factors related to over sedation, in accordance with the present disclosure.

FIG. 4 is a non-limiting example interface used in displaying data and monitoring factors related to over sedation, in accordance with the present disclosure.

FIG. 5 is a non-limiting example interface used in displaying data and monitoring factors related to over sedation, in accordance with the present disclosure.

FIG. 6 is a non-limiting example interface used in displaying data and monitoring factors related to over sedation, in accordance with the present disclosure.

### DETAILED DESCRIPTION

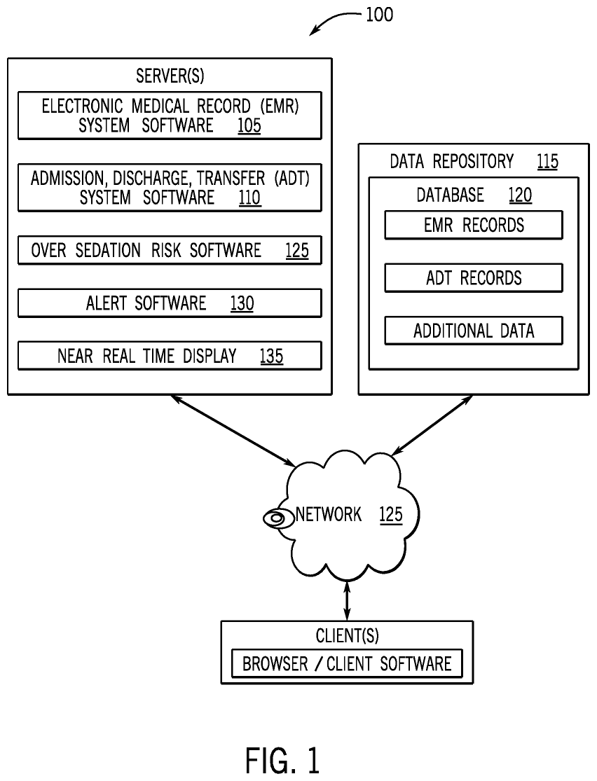

The disclosed system may be configured to execute several instructions within the software logic executed by one or more processors on one or more computing devices. These instructions may drive the system, to collect data, store the data within a data repository, and use that data to make decisions about whether or not to administer sedative medication. In general, as shown in FIG. 1, the over sedation alert and monitoring system 100 includes one or more Electronic Medical Record (EMR) software modules 105, one or more Admission, Discharge, Transfer (ADT) software modules 110, and a data repository 115, possibly including one or more databases 120.

Additional software modules within the disclosed system may include: one or more over sedation risk score/risk level calculation software engines or modules 125; one or more alert software modules 130; and one or more near real time display software modules 135.

The components of the over sedation alert and monitoring system 100 may be located on the same device, as shown in FIG. 1, including, but not limited to, a server, mainframe, desktop Personal Computer (PC), laptop, Personal Digital Assistant (PDA), telephone, mobile phone, kiosk, cable box, and the like. Alternatively, the components of the over sedation alert and monitoring system 100 may be located on separate devices connected by a network 125 (e.g., the Internet), with wired and/or wireless segments.

For purposes of this disclosure, the terms server or server computer may refer to any combination of the components of over sedation alert and monitoring system 100, running any of the algorithms for the software modules and/or software engines described herein. For example, servers may include one or more software modules or software engines, and the logic for their associated algorithms, executing on one or more computing devices, such as a server computer aggregating the data in the data repository 115, extracting data from the EMR 105 or ADT 110 systems described below, generating models or factors for determining a risk of over sedation, using the models to generate score or additional model calculations, making these determinations available through an interface available from the near real time display software 135, and/or using an API, or rendering and transmitting a user interface on a web page using a server-side graphical user interface module, and displaying the interface on a client computer using a client-side graphical user interface module, as non-limiting examples.

The server computer(s) may therefore execute the method steps within one or more of the disclosed algorithms by sending instructions, possibly in the form of compiled and executable software code for any of the disclosed software modules, to a processor on the server computer(s). The processor may then execute these instructions causing the server computer(s) to complete the disclosed method steps.

The data repository 115 may be configured to store data associated with the over sedation alert and monitoring system 100 The data repository 115 may be any sort of system capable of storing data, such as a relational database, a database management system, a hierarchical file, a flat file, and the like. The data repository 115 may be directly connected with any of the disclosed software modules, and/or to the server computers themselves, through various network configurations (e.g., wired, wireless, LAN, WAN, etc.). In one non-limiting example, the data may further include healthcare data associated with patients admitted to healthcare facilities.

The data repository 115 seen in FIG. 1, may be made up of a big data platform, sometimes referred to as a “data lake.” Data from any available enterprise-wide health information systems may be aggregated into this data repository 115. As a non-limiting example, the data platform may include Cloudera, implementing SAS and/or an instance of Hadoop. In this non-limiting example, Hadoop may provide open source, flat-file software that allows for the distributed processing of large datasets.

The disclosed system may further include an EMR system 105. The EMR system may comprise any combination of data and software logic used to capture any personal, documentation, clinical, and/or treatment data acquired as patients are admitted to, diagnosed, and treated within, a hospital or other healthcare facility. Typically, in order to access and analyze patient data, providers must access the EMR system 105. While this allows providers access to data information regarding patients, if the providers need this data in real time from the EMR system 105, they may access it as it is entered into the EMR system 105, but must do so manually.

The disclosed system may further include an Admissions, Discharge, and Transfer (ADT) system 110. The ADT system may be configured to track multiple types of data, including the reasons that patients were admitted, as well as admission diagnosis information, access to data from nursing notes, patient vitals, etc. The ADT system may then store the collected data in standard discreet values. The ADT system also includes records noting when and why patients were discharged, and/or transferred. Like the EMR system 105 above, typically, in order to access and analyze patient data, providers must access the ADT system 110. While this allows providers access to data information regarding patients, if the providers need this data in real time from the ADT system 110, they may access it as it is entered into the ADT system 110, but must do so manually.

By contrast, the disclosed embodiments include a near real time display system 135, allowing providers to instantly have access to the data within the EMR system 105, ADT system 110, and any additional data available from any available enterprise-wide health information systems stored in the data repository 115.

No limitations should be placed on the type of interface to be used by the disclosed embodiments. As non-limiting examples, interfaces may include: a graphical user interface (GUI) that is displayed by a browser or other client-side software; an API model, in which an API receives one or more Remote Procedure Calls (RPC) from a user and/or additional software, and generates response data to be used in any possible data format (e.g., data elements populated into another app or web technology), or into data repository 115; or populating a Computer Telephony Integration (CTI) within a call center, as non-limiting examples.

In one non-limiting example, the over sedation alert and monitoring system 100 may include multiple interfaces that may be accessed and manipulated by multiple users simultaneously, such as an API model, in which users, other devices, and/or software performing RPCs access the one or more available interfaces. Similarly, various instances of the interface may be accessed on multiple browsers or using an RPC from additional software, possibly client-side software. Additionally, the over sedation alert and monitoring system 100 may be optionally connected to data repository 115 and/or multiple databases 120. The over sedation alert and monitoring system 100 may connect to the data repository 115 and/or databases 120 physically and/or over a network 150, for example.

In embodiments that include displaying the over sedation user interface on a browser on a client machine, the browser may be configured to display the dashboard on a user interface, which may be a graphical user interface (GUI) associated with the over sedation alert and monitoring system 100. Those skilled in the art, having benefit of this detailed description, will appreciate that there will be many ways in which a GUI may be viewed other than using a browser

To provide a centralized data repository providing all accessible data, the disclosed system may provide for software that receives the data via the EMR system 105 or ADT system 110, or any other enterprise-wide health information systems, and automatically drives or pushes the data from the multiple source systems to the data repository 115. As the data is updated, the data may therefore also be aggregated and updated in real time in the data repository 115. As non-limiting examples, the data received and aggregated within the data repository 115 may include provider documentation, clinical data, surgical history, and medication data (e.g., medications given). Additional general data may include notes or other user input demonstrating the reason for the patient's visit to and/or admission to the hospital or healthcare facility, and any additional insights that the provider may have.

As non-limiting examples, provider documentation, possibly gathered when the patient is admitted, may include: general data; patient personal data (e.g., patient's name, age, ethnicity, address, phone number, insurance, emergency contacts, etc.); social history; smoking history; notes from the admitting provider; a patient's reason for visiting the healthcare facility, or associated problems; doctors' or nurses' notes regarding the patient's visit; nursing or other provider general documentation; notes regarding a patient's mental status; a doctor or other provider's diagnosis; provider's observations; medical history; placement of an order by the doctor or other provider; and the like.

As non-limiting examples, clinical data may include initial or ongoing examinations and/or observations, such as: vital signs determined by a doctor or on-site equipment generally; patient's temperature; patient heart rate; patient blood pressure; patient pulse ox (pO2); patient respiratory rate; patient glucose level; patient white blood cell count; patient platelet count; patient bilirubin count; patient lactate level; patient creatinine level; patient INR level; patient PTT; patient renal and hepatic function (e.g., whether renal or hepatic function is impaired); patient POSS score; patient RASS score; patient MOSS score; providers' diagnoses; and the like.

Non-limiting examples of patient surgical history may include: patient surgery case; patient surgery case procedure; Abdominal or Thoracic surgery performed on the patient; patient anesthesia time (e.g., has anesthesia been administered greater than 3 hours previously, or in the last 24 hours); and the like.

As non-limiting examples medication data may include: medications given; opioid or benzodiazepines medication administration; diagnoses that prompted the medication being prescribed, medication documentation; whether a sedative was given less than 2 hours ago; whether anesthesia time was greater than 3 hours or within the last 24 hours; and the like.

It should be noted that although the non-limiting examples above are grouped, these groups are for example purposes only. Any of the data above may be included in any group. Furthermore, the list of data within the groups is non-limiting. The disclosed embodiments may utilize any data within the data repository 115 to analyze patient data and provide the alerts, conclusions, and displayed data described within the disclosed embodiments.

Returning to FIG. 1, the disclosed embodiments may further include one or more software engines and/or software modules which may, as non-limiting examples, include an over sedation risk score/level calculation software 125 configured to determine a patient's risk of over sedation, according to the data for each patient stored in the data repository 115, and generate alerts for those patients in danger of being over sedated, as described in more detail below.

FIG. 1 also demonstrates an alert software 130 that may be configured to transmit and display alerts to providers, as certain criteria are identified, as disclosed in more detail herein. In some embodiments, the alerts software 130 may be configured to generate a text-based alert, which may be transmitted to a provider's personal mobile device or email account. In some embodiments, the provider may access a personal account to provide settings for when and how the criteria or threshold is reached, and the alert should be transmitted. The location of the providers is irrelevant to receiving the alerts; the alert may be available through text, GUI, phone message, etc., which may be set by the user.

In other embodiments, the alerts software 130 may be configured to generate and transmit an alert for display on the near real time display 135 described below. The alert may be triggered according to the algorithms and/or method steps described in more detail below.

As further seen in FIG. 1, the disclosed system may further include near real time display software 135, including logic to constantly and/or intermittently select data from the data repository 115, execute the algorithms and/or method steps described herein, and display the results on one or more client devices, possibly operated by the providers, and coupled to the disclosed system. As with the alerts described above, regardless of the providers' location, they may still have access to the information within the data repository 115 through use of the real time display software 135.

The disclosed system may be constantly updating the data repository 115, by pulling data from the EMR software 105, ADT software 110, or other associated systems for all patients for whom records exist. The near real time display software 135 may then be updated at regular intervals to reflect any new data received. As a non-limiting example, this data may be updated every 10-20 minutes.

In some embodiments, the software engines and/or modules may analyze the data received in order to implement machine learning algorithms to test the existing data and data logic within the system. As a non-limiting example, the data returned to the data repository 115 may be used as input, determined factors, and parameters to be input into a machine learning algorithm, allowing the system to be self-learning, so that, for example, thresholds determined may be adjusted based on the machine learning, or that false positives and false negatives may be detected to improve both alert performance and clinician confidence. Likewise, the risk scores and risk levels may be adjusted dynamically based on data analysis to determine where the thresholds should exist to define the patient's status as low, moderate, or high risk, which, in turn, may be used to update the models used to determine the patient's risk score, by pulling additional data to determine the outcomes, explain the models, and make the models more sensitive and specific.

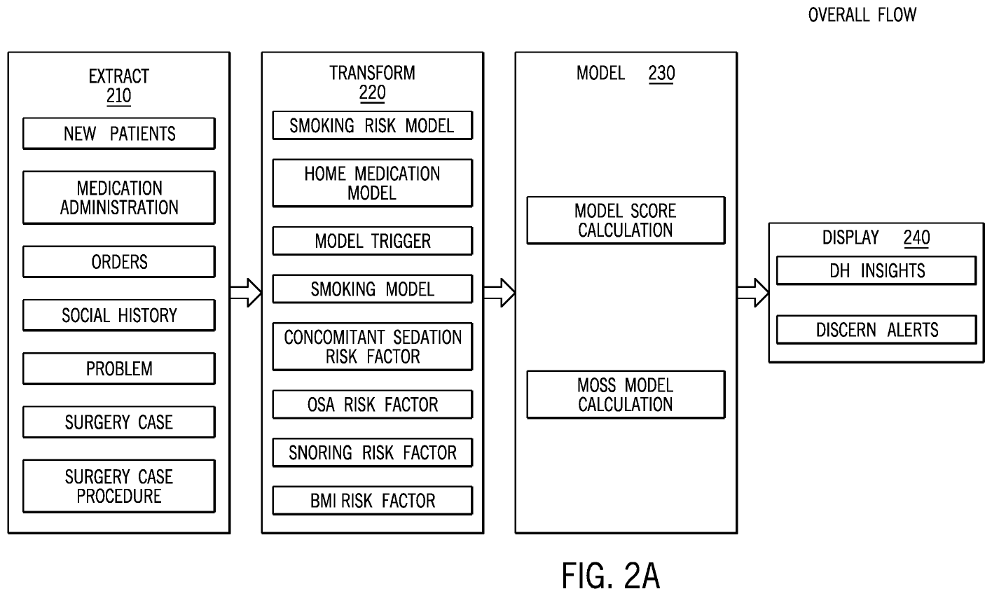

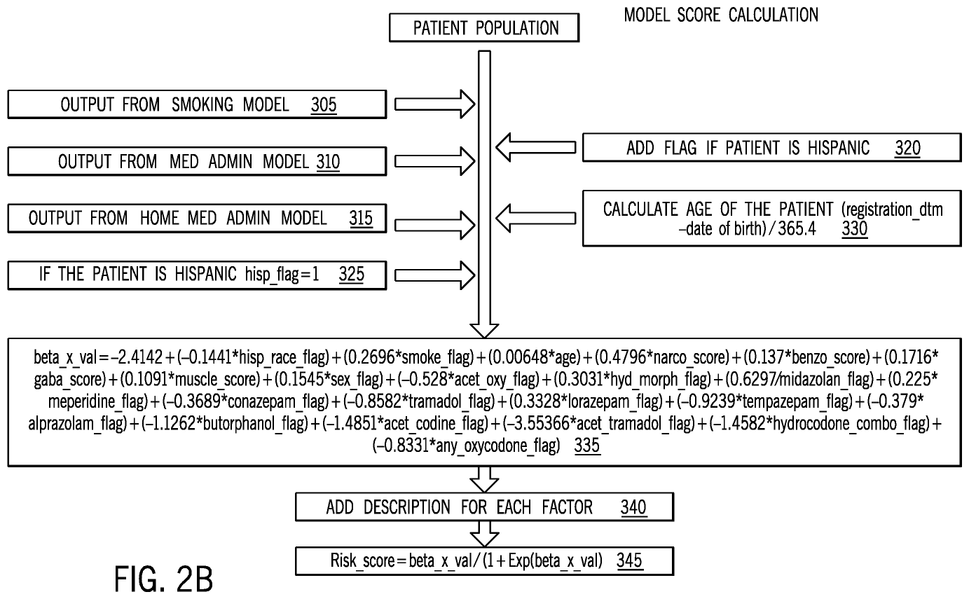

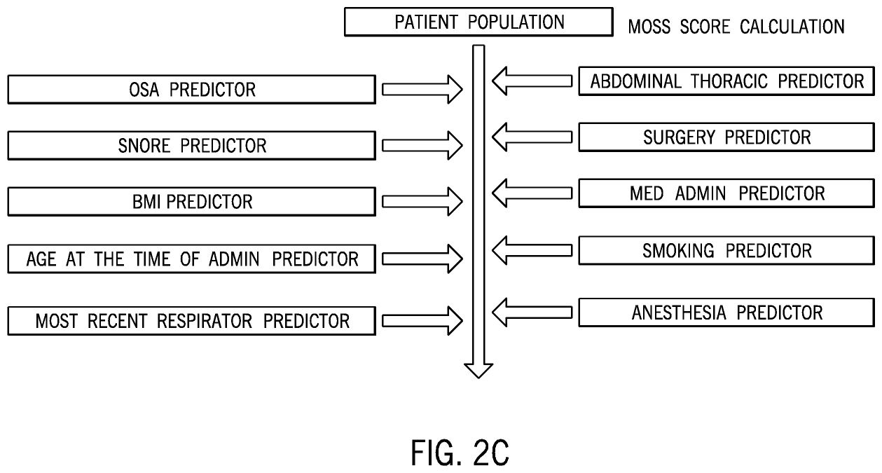

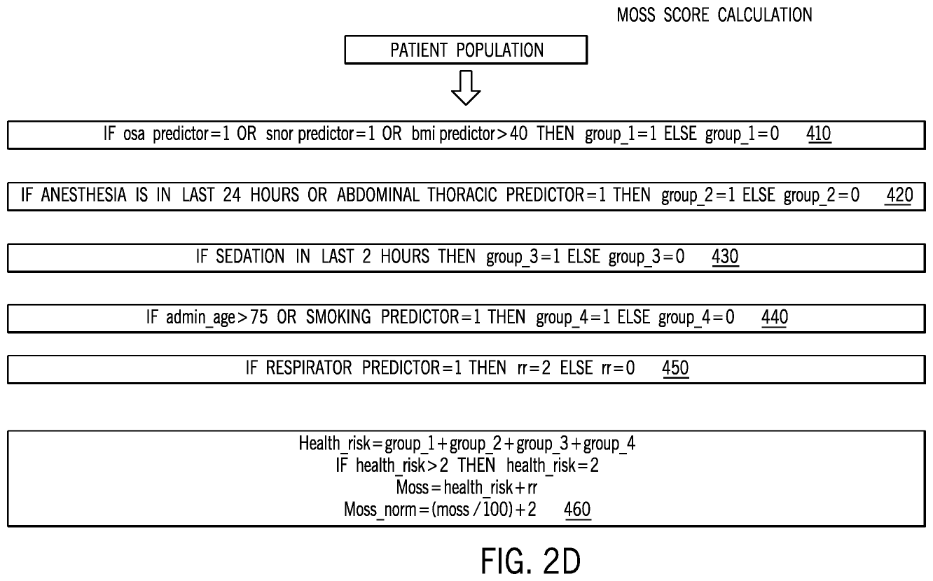

Turning now to FIGS. 2A-2D, patients may be admitted to a hospital, possibly as emergency department patients or an in-patient scenario, and may be observed and monitored during their stay. Providers may interview the patients as they are admitted to gather information, take various vitals, make general observations, run labs, make diagnoses, etc., and enter this data into the disclosed system, As non-limiting examples, the disclosed software and data repository 115 may process the received patient data at the time of admission, and generate data, possibly in the form of data records, for input into the EMR 105 or ADT 110 systems. As described above, to avoid this data being localized at the point of entry, this data may be automatically transmitted from the terminal to be stored as aggregated data within the data repository 115.

The aggregated data within the data aggregation 115 may then be available to the over sedation alert and monitoring system 100, which may calculate and identify the over sedation risk score and/or and generate any associated alerts for any admitted patients. In some embodiments, the criteria for calculation of the risk score may include criteria based on established and dynamic risk factors. The disclosed system may include one or more algorithms done on the data warehouse side based upon physiologic signs or symptoms or results that are available at that time a new patient is admitted.

In some embodiments, the algorithms executed in the disclosed system run parallel to, but outside of the EMR 105 and ADT 110 systems, then feed seamlessly back into the alert software 130, presenting the aggregated data to providers using the near real time display 135.

Once a new patient has been admitted, the data collected in association with the admission and the patient may be captured and input into the EMR system 105 and/or the ADT system 110. In addition, previous information from previous healthcare facility visits may have been input into the disclosed system and stored in the data repository 115 as medical records, notes, observations, previous medications administered, etc.

Turning now to Step 210, after the data has been input into and aggregated within the data repository 115, the disclosed system 100 may be further configured to extract data from the data repository 115, and pull that data in real time, at regular intervals (e.g., every 10-20 minutes). As non-limiting examples, the data extracted for use in the disclosed system 100 may include: patient current and historical vitals, patient health history, patient age, patient sex, patient weight, additional personal patient factors (ethnicity, language spoken, etc.), patient current medications, drugs taken by the patient, and the like.

Non-limiting examples of additional data extracted, seen in Step 210 of FIG. 2A, may include: new patient data; the administration of medication for the patient, orders placed in association with the patient, social history of the patient, one or more problems identified by or for the patient, a surgery case history for the patient, and one or more surgery case procedures.

The system may then aggregate the data extracted from the data repository 115, and any additional disclosed systems, and pull this data into a cloud platform, as needed. As additional data is aggregated and pulled into this cloud platform, the disclosed system 100 may continue to identify any changes to the data, and by extension, to the patient, to update the data and execute the algorithms disclosed herein with greater accuracy.

Returning to FIG. 2A, step 220 includes a transformation of the data extracted in step 210. The system may execute an algorithm during which the data extracted from the data repository 115 (possibly the data included in the non-limiting examples above) are used to generate, and/or are input into various models. Various risk factors are also established and analyzed during Step 220. As seen in this step, non-limiting examples of models generated and/or into which the extracted data may be input include: a model trigger; a smoking model; a smoking risk model; a medication model; and a home medication model.

As non-limiting examples, the smoking data extracted, associated with each patient (e.g., whether the patient is a smoker, the amount that the patient smokes, etc.) may be used to generate the smoking model and/or may be input into the smoking model and the smoking risk model used in calculating an over sedation risk score and/or level for each respective patient. Similarly, medication administrated data extracted, associated with each patient, may be used to generate the medication model, and/or input into the medication model, and/or the home medication models used in calculating an over sedation risk score and/or level for each respective patient. Data extracted from the data repository 115 may then be input into these models to generate a model score calculation, discussed in more detail below.

The data extracted from the data repository 115 may further be used to identify a plurality of risk factors for calculating an over sedation risk score and/or level for each patient. In some embodiments, these factors may be divided into dynamic risk factors and non-dynamic risk factors.

Dynamic risk factors may include risk factors recognizing that certain surgical procedures, and associated data, put the patient at higher risk. Thus, the algorithm includes built in logic that takes certain risk factors (e.g., certain surgical procedures, sedatives, anesthesia, etc.) into consideration in calculating the risk score or risk level.

As non-limiting examples, such dynamic risk factors may include a sedative given less than 2 hours previous to the current data extraction, an anesthesia time greater than 3 hours, and/or an anesthesia time less than 24 hours previous to the criteria data extraction. As noted above, certain surgical procedures may also put patients at higher risks, due to metrics in the algorithm that reflect the severity of the surgical procedures, according to data extracted from the EMR system 105. Thus, abdominal or thoracic surgery may create a higher risk score or risk level, due to the risks inherent in these surgical procedures.

Non-dynamic risk factors may include any risk factors that are not considered dynamic risk factors. Non-limiting examples of non-dynamic risk factors may include the patient's age (e.g., more than 75 years old), whether or not the patient is a smoker, whether or not the patient has been diagnosed with obstructive sleep apnea (OSA), whether or not the patient snores, the patient's Body Mass Index (BMI), and the like. Thus, in Step 220, the transformation within the overall flow may include, as non-limiting examples, an OSA Risk Factor, a Snoring Risk Factor, and a BMI Risk Factor.

Additional non-limiting examples of additional non-dynamic risk factors may include a Concomitant Sedation Risk Factor, diagnosis and medication documentation, co-morbidity, additional data from the patient history, a determination of whether or not a patient's renal and/or their hepatic functions have been impaired, or indications of over sedation related to renal insufficiency in older patients. In cases where dynamic or non-dynamic risk factors are present, the healthcare professionals may receive alerts to more closely monitor these patients, as described in more detail herein.

In Step 230 of FIG. 2A, the disclosed system may use any combination of the models and risk factors extracted and transformed in Steps 210 and 220 respectively, to calculate a risk score and/or risk level for each of the patients for whom records exist in the data repository 115. As non-limiting examples demonstrated in Step 230, and FIGS. 2B-2D, the risk score and/or risk level of each patient may be calculated using any combination of a Model Score Calculation, and/or a MOSS Model Calculation.

Turning now to FIG. 2B, a model score may then be calculated. The disclosed algorithm may receive patient population data from the data repository 115 after the extraction performed in Step 210 (e.g., every 15 minutes). This data may be used to generate and/or used as input into the models demonstrated in Step 220.

The output from these models may be used in the model score calculation seen in FIG. 2B. As non-limiting examples, for each patient for which the risk score and/or risk level is calculated, the disclosed system 100 may use the extracted data as input into the smoking model, and the model may output, as seen in Step 305, output from the smoking model, used in the model score calculation. Similarly, the disclosed system may calculate the model score by using the extracted data related to medication administration as input into the medication administration model, and/or the home medication administration model, and the model may output, as seen in Step 310 and 315, output from the medication administration model or the home medication administration model respectively.

The algorithm may also determine, from the extracted and transformed patient population data, whether the patient is Hispanic in Step 320, and if so, add a Hispanic flag to the database in Step 325(If the patient is Hispanic, hisp_flag=1).

Finally, in Step 330, the algorithm may calculate the age of the patient by subtracting the patient's date of birth from the date of the patient's admit/registration, divided by the number of days in a year, 365.4 ((registration_dtm−date of birth)/365.4).

Using the output from the various models, as well as the Hispanic flag and the patient's age, the algorithm may calculate a variable, beta_x_val, to be used in the model score calculation of the patient's risk score and/or risk level calculation.

As seen in Step 335, the beta_x_val variable may be calculated by multiplying various factors and database flags by a predefined numeric value, according to the following formula and/or calculation:

beta_x_val=−2.4142+(−0.1441*hisp_race_flag)+(0.2696*smoke_flag) +(0.00648*age)+(0.4796* narco_score)+(0.137*benzo_score)+(0.1716* gaba_score)+(0.1091*muscle_score)+(0.1545*sex_flag)+(−0.528*acet_oxy_flag)+(0.3031*hyd_morph_flag)+(0.6297*midazolam_flag)+(0.225*meperidine_flag)+(−0.3689*conazepam_flag)+(−0.8582*tramadol_flag)+(0.3328*lorazepam_flag)+(−0.9239*tempazepam_flag)+(−0.379*alprazolam_flag)+(−1.1262*butorphanol_flag) +(−1.4851*acet_codine_flag)+(−3.5366*acet_tramadol_flag)+(−1.4582* hydrocodone_combo_flag)+(−0.8331*any_oxycodone_flag)

As an interim step, in Step 340, the disclosed system may add a description for each factor used in the calculation of beta_x_val in step 335.

Finally, in Step 345, the disclosed system 100 may then make the final calculation of the risk score, by dividing beta_x_val by 1 plus Exp. multiplied by beta_x_val (Risk_score=beta_x_val/ beta_x_val)).

Returning to FIG. 2A, in Step 230, the system may further use the extracted and transformed data from Steps 210 and 220 respectively, to calculate a Michigan Opioid Safety Score (MOSS), an additional and/or alternative algorithm to calculate risk of over sedation.

FIG. 2C and 2D demonstrate the steps in calculating such a MOSS score. As with the model score calculation seen in FIG. 2B, the disclosed algorithm may extract and receive patient population data from the data repository 115 regularly (e.g., every 15 minutes), and generate and/or use the extracted data and/or factors disclosed above as input into the models. As seen in FIG. 2C, the extracted and transformed patient population data may be used to identify multiple predictors for the algorithm, related to the extracted and transformed data.

As non-limiting examples seen in FIG. 2C, the MOSS score algorithm may identify, from the extracted and transformed patient population data: an OSA Predictor; a Snore Predictor; a BMI Predictor; an Age at the Time of Administration Predictor; a Most Recent Respirator Predictor; an Abdominal Thoracic Predictor; a Surgery Predictor; a Med Admin Predictor; and an Anesthesia Predictor.

As seen in FIG. 2D, the algorithm may then execute several conditional statements to determine a score for each of several groups, and a sum of the group scores is then used to determine a patient's health risk.

The conditional statements may use the predictors from FIG. 2C within conditional statements to determine four sub-scores for each of four groups related to: the OSA predictor, snore predictor or the BMI predictor; the anesthesia predictor (in the last 24 hours), and the thoracic predictor; the sedation of the patient in the last 2 hours; and the age at the time of admin predictor, respectively, as well as a sub-score related to respiratory rate, based on the respirator predictor. These sub-scores and groups may be used to determine a final health risk score.

Specifically, as seen in Step 410 of FIG. 2D, if the OSA predictor is equal to one (OSA predictor=1), or the snore predictor is equal to 1 (snore predictor=1), or the BMI Predictor is greater than 40 (BMI Predictor>40), the algorithm may assign group one of the four groups a value of 1. However, if none of these conditions are true, the algorithm may assign group one of the four groups a value of 0 (If osa predictor=1 or snore predictor=1 or bmi predictor>40 then group_1=1 else group_1 =0).

In Step 420, if the anesthesia predictor determines that the administration of anesthesia occurred in the last 24 hours or the abdominal thoracic predictor is equal to 1 (thoracic predictor=1), the algorithm may assign group two of the four groups a value of 1. However, if none of these conditions are true, the algorithm may assign group two of the four groups a value of 0 (If anesthesia is in last 24 hours or abdominal thoracic predictor=1 then group_2 =1 else group_2 =0).

In Step 430, if there has been sedation administered to the patient in the last 2 hours, the algorithm may assign group three of the four groups a value of 1. Otherwise, the algorithm may assign group four a value of 0 (If sedation in last 2 hours then group_3=1 else group_=0).

In Step 440, if the patient's age is over 75 (admin_age>75), or if the smoking predictor is equal to 1 (smoking predictor=1), the algorithm may assign group four of the four groups a value of 1. However, if neither of these conditions are true, the algorithm may assign group four a value of 0 (If admin_age>75 or smoking predictor=1 then group_4=1 else group_4=0).

Finally, in step 450, if the respirator predictor is equal to 1 (respirator predictor=1), then the respiratory rate (rr) may be set equal to 2 (rr=2), otherwise the algorithm may set the respiratory rate variable to 0.

In Step 460, the final MOSS score may then be calculated. First, the algorithm may create a Health_risk variable, and assign it the value of Health_risk=group_1+group_2+group_3+group_4.

The algorithm may then determine whether the health_risk variable is greater than 2, and if so, then may set the health_risk variable equal to 2 (if health_risk>2 then health_risk=2).

The algorithm may then calculate the MOSS score by generating a sum of the value stored in the health_risk variable added to the rr variable representing the respiratory rate (MOSS=health_risk+rr).

Finally, a normalized MOSS score may be calculated by dividing the MOSS score by 100 and adding 2 (MOSS_norm=(MOSS/100)+2).

This process may be repeated at a regular interval (e.g., every 10-20 minutes).

The disclosed embodiments may then use the calculation of the model score and/or the MOSS Model calculation to identify a risk level for the patient, and may store this risk level in association with the patient's records in the data repository 115. Each patient's risk level may be determined by comparing the final risk score calculated above with predefined thresholds, possibly stored in the data repository 115, or within the logic of the disclosed software, to assign a patient a specific risk level, as described below.

As non-limiting examples, in embodiments in which the MOSS model and score is used: if the MOSS score for a patient is less than 2, the patient may be assigned a low risk level; if the MOSS score for a patient is less than equal to 2, the patient may be assigned a moderate risk level; and if the MOSS score for a patient is greater than 2, the patient may be assigned a high risk level.

As a non-limiting example, the risk levels may include a safe level, a concern level, and a caution level. The safe level may be correlated with a low over sedation risk score, the concern level may be correlated with a moderate over sedation risk score, and the caution level may be correlated with a high over sedation risk score.

The databases 120 or the software logic within the software engines and/or modules in the disclosed embodiments may store a plurality of recommendations associated with each of the risk levels. As non-limiting examples, a safe level may be associated with a text message that a provider's patient is at low risk of over sedation. A concern level may be associated with a text message that a provider's patient is at a moderate risk of over sedation, and a recommendation to check vitals, get a POSS score for the patient, and to consider continuously monitoring the patient's SPO2 and/or respiratory rate, and a caution level may be associated with a text message that a provider's patient is at a high risk of over sedation, and a recommendation to check vitals, get a POSS score for the patient, and to consider continuously monitoring the patient's SPO2 and/or respiratory rate.

Once the risk level for each patient has been determined, the disclosed system may select all content for the associated recommendation from the data repository 115 and/or software logic, and generate a recommendation content, using the selected content.

The disclosed embodiments may then use the calculation of the model score and/or the MOSS Model calculation, in combination with the risk scores and risk levels to determine whether or not to generate and display an alert to the providers, including the generated recommendation content. This recommendation content may include an explanation of the reason for the risk, the risk factors, the patient's risk level, and recommended instructions within the content on how to proceed.

In some embodiments, the disclosed embodiments may determine that an alert should be generated and displayed if the user's risk score is above a certain threshold. As non-limiting examples, in some embodiments, an alert may be generated if the user is at a concern or a caution level, and/or if the user has a moderate or high risk score, respectively.

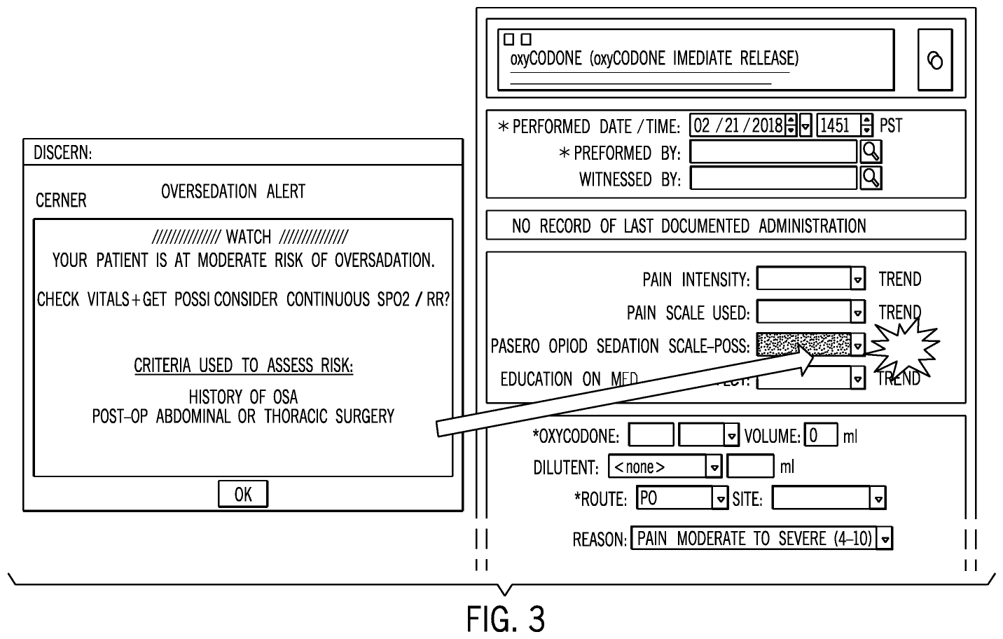

Turning now to FIG. 3, if the system determines that the risk level and/or risk score is greater than the threshold, the disclosed system may generate an alert for display to the user. As seen in FIG. 3, this alert may include a popup window, and the content of this popup window may include the content described above. Namely, the content of the over sedation alert may include a title, informing the user that the alert has been triggered, the risk level and/or the risk score for the patient (e.g., “Your patient is at MODERATE risk of OVER SEDATION”), and the recommendation content associated in the database 120 or data logic with the patient's risk score and/or risk level.

In some embodiments, such as that seen in FIG. 3, the alert may also display the criteria used to assess the risk. To accomplish this, the disclosed system 100 may log, as it is determining the risk score and/or risk level for the user, the extracted data associated with the user, and the models, model output, factors, and predictors from the method steps and calculations disclosed above, and display the most relevant factors in determining the moderate or high risk levels or scores within the content of the alert (e.g., “History of OSA, Post-op abdominal or thoracic surgery”).

As seen in FIG. 3, once the alert has been displayed, the user may click a button, acknowledging that they have seen the alert. On pressing the button, the disclosed system 100 may automatically open any related GUI windows, receiving documentation and input from the providers that responded to the alert. As a non-limiting example, in FIG. 3, clicking on the “OK” button may cause the system to automatically launch a user interface for documenting the post-opioid POSS in a Medication Response Window.

In some of the disclosed embodiments, the alert may be generated and displayed, if the system has determined that a patient is at a moderate or high risk, as each provider logs into the system and accesses the user interface/dashboard described in detail below. Turning again to FIG. 2A, the disclosed system may update the current information at regular intervals, such as every 10-20 minutes, using the extract (Step 210), transform (Step 220), and model (Step 230) steps described above. Then, in Step 240, the disclosed system may display the alerts, where appropriate, as providers access the system, providing an opportunity for the provider to review the information available on the user interface and determine why the alert may have fired, to determine whether an additional dose of sedative or other medication is appropriate, as described in more detail below. However, additional embodiments could be imagined, in which there are “push” alerts, so that if a patient has a moderate or high risk score/level, the system automatically generates an alert for display on the user interface.

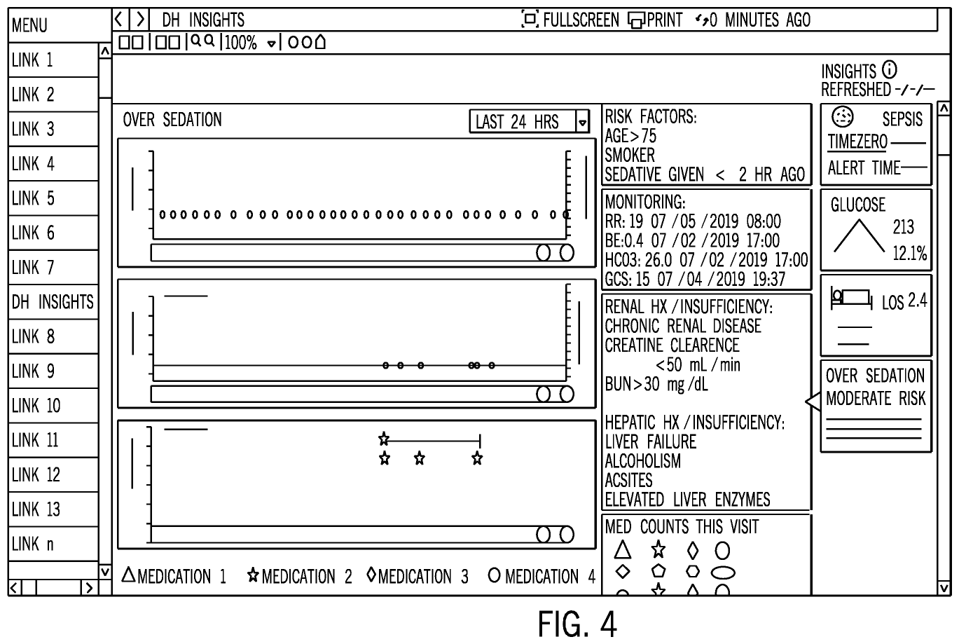

FIGS. 4-6 demonstrate non-limiting examples of the user interface or dashboard generated by the near real time display software 135 within the disclosed system, providing a quick visual for the providers to assess reasons for the alert firing and to track and monitor the patient's status, to more effectively respond to the information provided in determining whether or not to provide additional medication.

As the data in the data repository is updated at regular intervals, the near real time display 135 may populate, refresh, and/or otherwise synchronize the user interface with the data repository 115 to reflect this newly received data. As non-limiting examples, this displayed data may include any new medications administered, any changes in the patient's risk level, recently input POSS or RASS scores, and the like.

As demonstrated in FIGS. 4-6, the user interface may be accessible by, integrated into, and/or displayed as a portion of the EMR system. As a non-limiting example, the disclosed user interface may be accessed by selecting a menu item within the EMR system.

As seen in FIGS. 4-6, the dashboard may be made up of three synchronized timelines, divided into three synchronized rows representing various patient data as it relates to the timeline. These rows may include a Risk/Respiratory Rate row, a POSS/RASS score row, and a Medications Given row. The provider may therefore view the patient's risk score/respiratory rate, provider input, and/or various medications along the timeline to review the patient history, and draw conclusions from the displayed data. The timeline may be adjusted according to a user input into a user interface element, such as the dropdown menu seen in FIGS. 4-6.

As seen in FIGS. 4-6, the first of these three rows may include a visual representation of the over sedation risk score and/or level for the patient. The disclosed system 100 may execute the algorithms described above, and determine the risk score and/or risk level for the patient for whom the dashboard is being displayed. The risk score/level, determined at a regular interval, may be represented by a dot or dash shown along the timeline at the time the data was updated at a regular interval according to the calculations using the data from the data repository 115 as described above. These risk scores and/or levels may further be correlated according to the patient's respiratory rate, as reflected in FIGS. 4-6.

In some embodiments, the dot or dash along the timeline may be color coded to reflect the risk score or level. Similarly, as seen in FIGS. 4-6, the dots or dashes may be displayed in a vertical manner in a way that reflects whether the risk score is a low, moderate, or high risk. As a non-limiting example, risk scores determined along the timeline that are within a low risk score or level may be represented by a green dot or dash and/or at a lower vertical height in the timeline than those representing moderate or high risks. Risk scores determined along the timeline that are within a moderate risk score or level may be represented by a yellow dot or dash and/or at a medium vertical height, higher than low risk entries, but lower than high risk entries, and risk scores determined along the timeline that are within a high risk score or level may be represented by a red dot or dash, and may appear at a higher vertical height than those representing low or moderate risks.

As seen in FIGS. 4-6, the second of the three rows may include a visual representation of calculation of POSS scores or RASS scores. These opioid sedation scores may reflect the providers' clinical assessment of the patient, and reflect, and may be administered according to nationally known and standardized acceptable scores for over sedation risk. In addition, physiological signs may be used, at regular intervals, to contribute to the determination of the risk score. In some embodiments, such as those demonstrated in FIGS. 4-6, the two scores are displayed together because both scores may influence each other.

Thus, the disclosed embodiments may include a POSS score, which is part of the nurse's assessment, as well as physiologic signs that are calculated every 15 minutes to determine the patient's risk. The POSS score utilized in the disclosed embodiments may refer to the Pasero Opioid-induced Sedation Scale, a valid, reliable tool used to assess sedation when administering opioid medications to manage pain. The POSS is endorsed by The Joint Commission and the American Society for Pain Management Nursing to help prevent adverse opioid-related respiratory events.

The disclosed embodiments may further utilize a RASS score, the Richmond Agitation-Sedation Scale is a medical scale used to measure the agitation or sedation level of a person. It was developed with efforts of different practitioners, represented by physicians, nurses and pharmacists. The RASS can be used in all hospitalized patients to describe their level of alertness or agitation. It is however mostly used in mechanically ventilated patients in order to avoid over and under-sedation. Obtaining a RASS score is the first step in administering the Confusion Assessment Method in the ICU (CAM-ICU), a tool to detect delirium in intensive care unit patients. Thus, the disclosed embodiments may include a record of input regarding these two scores within the user interface, to reflect the historical input POSS and RASS scores.

As seen in FIGS. 4-6, the third of the three rows may include a visual representation of medications given to the patient along the timeline, giving providers insights via a visual record of what medications were given at what time.

The example embodiments shown in FIGS. 4-6 demonstrate a single resource that may demonstrate to providers, chronologically and longitudinally, a history of narcotics administered, sedation scores, and current risk, and how they affect one another, in order to make the determination of next steps for medication administration. By comparing the three rows, the providers may determine cause and effect relationships between medications given and the effect of those medications on the risk score, the patient's respiratory rate, high POSS and/or RASS scores. For example, if a medication was given to a patient, and their risk, respiratory rate, POSS, and/or RASS scores spiked shortly after the administration of the medication, the providers may determine visually from the user interface demonstrated in FIGS. 4-6 that the medication was the cause of the spike in the other scores. The providers may then visually identify trends from these correlations, so that they may determine, over time, that the administration and timing of certain medications create a greater risk for the patient.

As seen in FIGS. 4-6, the user interface may include additional user interface elements to provide the providers with additional data from the data repository. As seen in the non-limiting examples seen in FIGS. 4-6, the user interface may further include a panel displaying the risk factors used in determining the risk score for the patient. In these examples, the risk factors used to determine the risk score and/or risk level for the user may include an age over 75, the patient being a smoker, and a sedative given less than 2 hours previous. In the examples seen in FIGS. 4-6, the user interface may further include a panel displaying the results of monitoring the patient. In these non-limiting examples, the user interface panel may include the most recent level, and the time at which these metrics were determined. In the examples seen in FIGS. 4-6, the user interface may further include a panel displaying the current status of the patient's renal and hepatic health. In these examples, renal and hepatic insufficiencies may be displayed that affect the patient's risk factors, and other scores displayed in the user interface. In the examples seen in FIGS. 4-6, the user interface may further include a panel displaying the med counts for a patient during a particular visit. In these examples, a count of each of the medications may be displayed using the key for medications displayed at the bottom of the user interface in FIG. 4. In the examples seen in FIGS. 4-6, the user interface may further include a panel displaying the status of a patent with sepsis. In these examples, the panel may display the time zero for the patient, and a time for any associated alerts. In the examples seen in FIGS. 4-6, the user interface may further include a panel displaying the status of a patent's glucose levels. In the examples seen in FIGS. 4-6, the user interface may further include a panel displaying the status of a patent's length of stay, including the current length of stay, whether or not the patient is inpatient or outpatient, and the expected length of stay for the patient. In the examples seen in FIGS. 4-6, the user interface may further include a panel displaying the status of a patent's risk of over sedation, including the current risk, and the recommendations. In this example, the recommendation to the provider may include checking the patient's vitals, determining the patient's POSS, and considering continuous SPO2/RR monitoring.

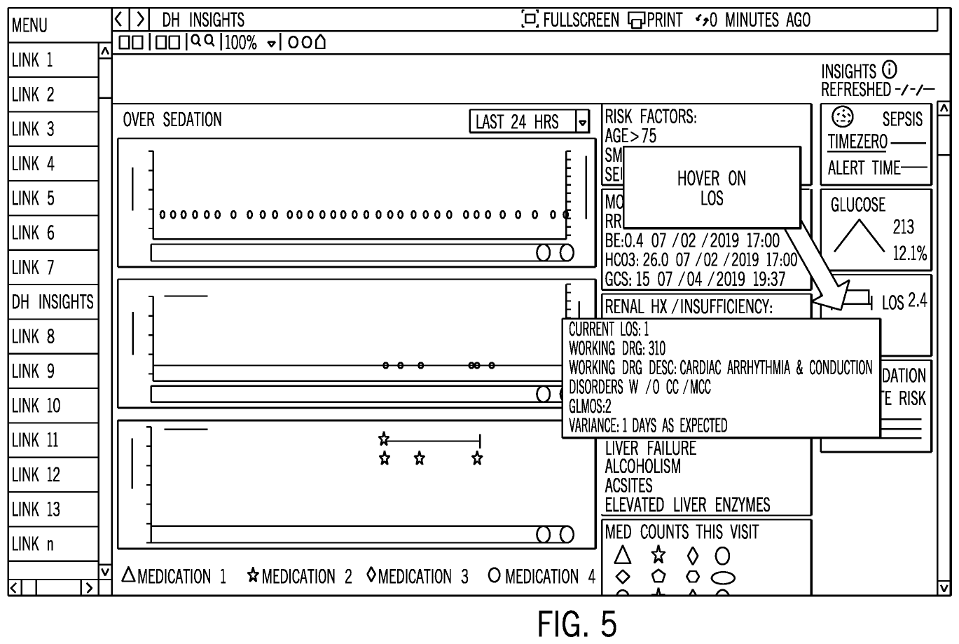

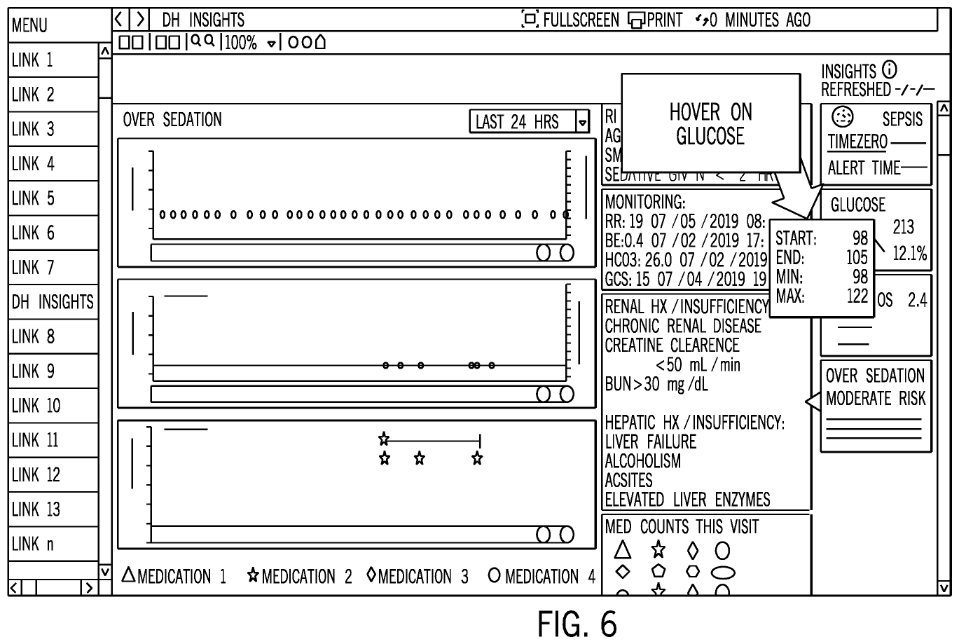

As seen in the non-limiting examples seen in FIGS. 5-6, the data available from the data repository may be accessible to the user by hovering over individual panels and/or elements within the user interface. For example, in FIG. 5, the provider may hover over the length of stay panel to display additional details about the patient's length of stay. Likewise in FIG. 6, the provider may hover over the glucose panel to display additional details about the patient's glucose.

These examples are non-limiting. The provider may hover over any element within the user interface to display additional details about that element.

A use case may demonstrate the utility of the disclosed embodiments. A provider, possibly at the beginning of a shift or during a transition of care, may access the disclosed user interface, possibly by accessing the provider's user account, and may access the over sedation software, possibly via a link in an EMR software, to view the status for a specific patient.

The disclosed system may have continually updated the data in the data repository 115, and run the calculations above to determine if the patient is at an appropriate risk score level to generate an alert. In some embodiments, the risk score/level may be determined after the provider scans the medication they intend to administer. If the patient is at an appropriate risk level, the alert may be displayed on the provider's monitor.

Before giving any additional medication, the provider may use their clinical decision making discretion in following the recommendations within the alert, specifically reviewing the frequency and timing of recent sedating medications (or all medications generally), determining a POSS and continuing to monitor the patient. If the provider does move forward with administering the medication, they may again determine a POSS after the administration of the medication.

The present invention has been described in terms of one or more preferred embodiments, and it should be appreciated that many equivalents, alternatives, variations, and modifications, aside from those expressly stated, are possible and within the scope of the invention.

1. A system comprising:
a data repository coupled to a computer network and storing a plurality of data aggregated in association with each of a plurality of patients, the plurality of data comprising:
a documentation data input by at least one healthcare professional in association with a patient in the plurality of patients;
at least one medication history record associated with the patient; and
at least one surgical history record associated with the patient;
a server, comprising at least one server hardware computing device coupled to the computer network and comprising at least one processor executing instructions within a memory which, when executed, cause the system to:
extract, from the data repository, a plurality of risk factor data, in the plurality of data, associated with at least one over sedation risk factor for the patient;
input the plurality of risk factor data into an over sedation risk model;
calculate, as output from the over sedation risk model, a risk score and a risk level for the patient;
responsive to the risk score exceeding a predetermined threshold, automatically generate an over sedation alert for the patient;
generate a Graphical User Interface (GUI) comprising:
a first GUI component displaying a synchronized timeline and a plurality of medications administered to the patient:
a second GUI component displaying the risk level for the patient at regular intervals along the synchronized timeline and;
a third GUI component displaying a sedation scale score for the patient monitored at regular intervals along the synchronized timeline.

2. The system of claim 1, further comprising a GUI popup window generated responsive to the risk score exceeding the predetermined threshold, and displayed on a client computer operated by at least one healthcare professional.

3. The system of claim 2, further comprising a content displayed on the GUI popup window, comprising:
a notification of the over sedation alert;
the risk level for the patient;
the at least one over sedation risk factor; and
a recommendation, associated in the data repository or in the instructions with the risk level for the patient.

4. The system of claim 1, further comprising a plurality of dots or dashes, displayed within the second GUI component, each of the plurality of dots or dashes representing the risk level for the patient along the synchronized timeline at a time that the risk level was determined.

5. The system of claim 4 wherein each of the plurality of dots or dashes is color-coded according to the risk level for the patient, wherein:
a low risk level is represented by a first color;
a moderate risk level is represented by a second color; and
a high risk level is represented by a third color.

6. A method comprising:
extracting, by a server, comprising at least one server hardware computing device coupled to the computer network and comprising at least one processor executing instructions within a memory, from a data repository coupled to the computer network, a plurality of risk factor data associated with at least one over sedation risk factor for a patient;
inputting, by the server, the plurality of risk factor data into an over sedation risk model;
calculating, by the server, as output from the over sedation risk model, a risk score and a risk level for the patient;
responsive to the risk score exceeding a predetermined threshold, automatically generating, by the server, an over sedation alert for the patient;
generating, by the server, a Graphical User Interface (GUI) comprising:
a first GUI component displaying a synchronized timeline and a plurality of medications administered to the patient:
a second GUI component displaying the risk level for the patient at regular intervals along the synchronized timeline and;
a third GUI component displaying a sedation scale score for the patient monitored at regular intervals along the synchronized timeline.

7. The method of claim 6, further comprising the step of deriving, by the server, the plurality of risk factors from a plurality of data aggregated within the data repository for each of a plurality of patients, the plurality of data comprising:
a documentation data input by at least one healthcare professional in association with the patient;
at least one medication history record associated with the patient; and
at least one surgical history record associated with the patient.

8. The method of claim 6, further comprising the step of identifying, by the server, within the plurality of risk factor data, at least one dynamic risk factor comprising:
a surgical procedure performed on the patient;
an anesthesia time for the patient greater than 3 hours and less than 24 hours; and
a sedative administered to the patient within the previous 2 hours.

9. The method of claim 6, further comprising the step of identifying, by the server, within the plurality of risk factor data, at least one non-dynamic risk factor comprising:
a first determination whether the patient smokes;
a second determination whether the patient snores;
an obstructive sleep apnea diagnosis for the patient;
an age for the patient; or
a body mass index metric for the patient.

10. The method of claim 6, wherein the sedation scale score comprises a Pasero Opioid-induced Sedation Scale score.

11. The method of claim 6, wherein the sedation scale score comprises a Richmond Agitation-Sedation Scale score.

12. A system comprising a server, comprising at least one server hardware computing device coupled to the computer network and comprising at least one processor executing instructions within a memory which, when executed, cause the system to:
extract, from a data repository coupled to the computer network, a plurality of risk factor data associated with at least one over sedation risk factor for a patient;
input the plurality of risk factor data into an over sedation risk model;
calculate, as output from the over sedation risk model, a risk score and a risk level for the patient;
responsive to the risk score exceeding a predetermined threshold, automatically generate an over sedation alert for the patient;
generate a Graphical User Interface (GUI) comprising:
a first GUI component displaying a synchronized timeline and a plurality of medications administered to the patient:
a second GUI component displaying the risk level for the patient at regular intervals along the synchronized timeline and;
a third GUI component displaying a sedation scale score for the patient monitored at regular intervals along the synchronized timeline.

13. The system of claim 12, wherein the server is further configured to generate a GUI popup window, for display on a client computer operated by the healthcare professional, responsive to the risk score exceeding the predetermined threshold, and displayed on a client computer operated by at least one healthcare professional.

14. The system of claim 13, wherein the server is further configured to generate, for display on the GUI popup window:
a notification of the over sedation alert;
the risk level for the patient;
the at least one over sedation risk factor; and
a recommendation, associated in the data repository or in the instructions with the risk level for the patient.

15. The system of claim 12, wherein the server is further configured to generate, for display on the second GUI component, a plurality of dots or dashes, each representing the risk level for the patient along the synchronized timeline at a time that the risk level was determined.

16. The system of claim 15 wherein each of the plurality of dots or dashes is color-coded according to the risk level for the patient, wherein:
a low risk level is represented by a first color;
a moderate risk level is represented by a second color; and
a high risk level is represented by a third color.

17. The system of claim 12, wherein the server is further configured to:
select the plurality of risk factor data or a plurality of additional data within the data repository;
input the plurality of risk factor data or the plurality of additional data as input, determined factors, or parameters, into a machine learning algorithm;
receive, as output from the machine learning algorithm:
at least one adjustment to the predetermined threshold;
an identification of at least one false positive or at least one false negative; or
a dynamic adjustment to the risk model, the risk score, or the risk level for the patient.

18. The system of claim 12, wherein the at least one over sedation risk factor includes:
at least one renal or kidney factor indicating whether a renal or kidney function for the patient is impaired;
at least one hepatic or liver factor indicating whether a hepatic or liver function is impaired; or
at least one indication of renal insufficiency in association with the patient.

19. The system of claim 12, wherein the GUI further includes a chronological and longitudinal display of:
a history of at least one medication administered, within the first GUI component;
at least one risk score associated with, and indicating an effect of, the at least one medication administered;
at least one trend associated with the first GUI component, the second GUI component, and the third GUI component.

20. The system of claim 12, wherein the server is further configured to:
receive, from within an electronic medical record (EMR) software application, a first user input selecting a link to the GUI; and
display the GUI within an embedded display within the EMR software application.
